(19) United States
(12) Patent Application Publication
(10) Pub. No.: US 2018/0361123 A1
(43) Pub. Date: Dec. 20, 2018

(54) CATHETER GUIDE TUBE DEVICE

(71) Applicant: Allen D. Seftel, Cherry Hill, NJ (US)

(72) Inventor: Allen D. Seftel, Cherry Hill, NJ (US)

(21) Appl. No.: 15/524,109

(22) PCT Filed: Mar. 11, 2016
(86) PCT No.: PCT/US16/21927

§ 371 Date: Dec. 1, 2017

(51) Int. Cl. A61M 25/06

(52) U.S. Cl. CPC A61M 25/0662; A61M 5/19; A61M 2210/1096; A61M 2210/1082

(57) ABSTRACT

A catheter guide tube device (10) to assist in the insertion of a catheter (11) through a genital urethary system to a desired location within a patient's body. The catheter guide tube device (10) includes a catheter (11), a catheter guide tube (14) being attached to the catheter, and a catheter injection system (26) to regulate and control a flow of liquid through the catheter.

### TECHNICAL FIELD OF THE INVENTION

The present invention relates generally to an improved catheter guide tube device. More specifically, the present invention relates to an improved catheter guide tube device used to administer a medication at a desired location within a patients body where a surgical wire is inserted.

### BACKGROUND OF THE INVENTION

The kidneys are a pair of organs that lie in the back of the abdomen on each side of the vertebral column of a mammalian patient, such as a human. The functions of the kidney can be summarized under three broad headings: a) filtering blood and excreting waste products generated by the body's metabolism, b) regulating salt, water, electrolyte and acid-base balance, and c) secreting hormones to maintain vital organ blood flow. Without properly functioning kidneys, a patient will suffer water retention, reduced urine flow, and an accumulation of waste toxins in the blood and body.

Processes and arrangements of application catheters are used to administer a medication at a desired location, with the objective of achieving an optimum action. This idea of local application underlies the idea of achieving better results with a high local concentration of the active ingredient and fewer systemic side effects. Many different medical device systems and methods have been previously disclosed for locally delivering fluids or other agents into various body regions, including body lumens such as vessels, or other body spaces such as organs or heart chambers. Local “fluid” delivery systems may include drugs or other agents, or may even include locally delivering the body's own fluids, such as artificially enhanced blood transport (e.g. either entirely within the body such as directing or shunting blood from one place to another, or in extracorporeal modes such as via external blood pumps etc.) A wide variety of catheters are available for insertion into the body for introduction or withdrawal of fluids. Catheters are flexible tubes designed to access various bodily organs, such as the kidneys, by insertion into the urethra.

### SUMMARY OF THE INVENTION

According to an embodiment of the present invention, there is disclosed a catheter guide tube device to assist in the insertion of a catheter at a desired location. The catheter guide tube device is characterized by a catheter, a catheter guide tube attached to the catheter, and a catheter injection system to regulate and control a flow of liquid through the catheter.

According to another embodiment of the present invention, there is disclosed a method for inserting a catheter at a desired location in a patient's body. The method includes inserting a surgical wire into a patient's body whereby a first end of the surgical wire is placed adjacently to a desired location within the patient's body adjacent to where the catheter is to be placed and a second end of the surgical wire remains external to the patient's body. Then, inserting the second end of the surgical wire into a first distal open end of a catheter guide tube attached to a catheter. Finally, feeding the catheter guide tube onto the surgical wire, wherein the first distal open end of the guide tube and a distal end of the catheter reaches the desired location within the patient's body.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a side three-dimensional view of the catheter guide tube device with a surgical wire in the guide tube, in accordance with the present invention.

FIG. 2 is a side three-dimensional view of the catheter guide tube device, in accordance with the present invention.

FIG. 2A is a cross-sectional view through line 2-2 of FIG. 2, illustrating the guide tube and catheter, in accordance with the present invention.

FIG. 3 is a three-dimensional view of the catheter guide tube device entering the genital urethary system of a patient's body, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

In the field of urology, it is sometimes necessary to enter the urinary tract or other passage of the human body in order to provide location specific medical treatment. A catheter 11 is typically a long, thin tube inserted through a patient's skin to reach the desired location, such as a kidney. The catheter 11 is connected at one end (commonly referred to as its proximate end) to a blood or saline bag, a blood pump or other medical device external to the body. It is often problematic to precisely insert the catheter at a highly specific location. The catheter guide tube device 10 of the present invention includes a catheter 11 in combination with a catheter guide tube 14 and is designed to assist in the insertion of the catheter into the body so that the end of the distal end of the catheter can be disposed at a specific desired location.

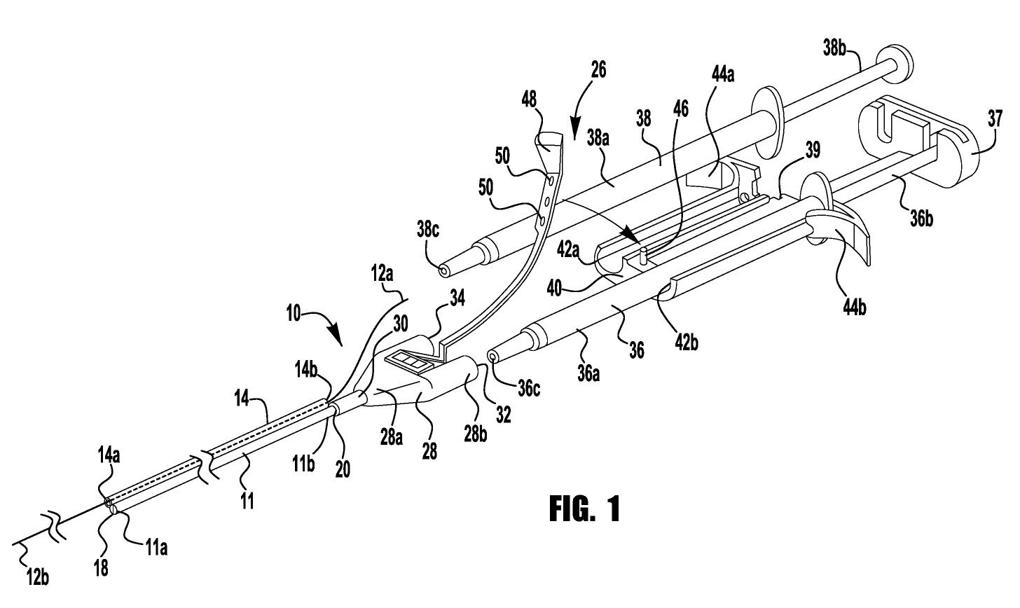

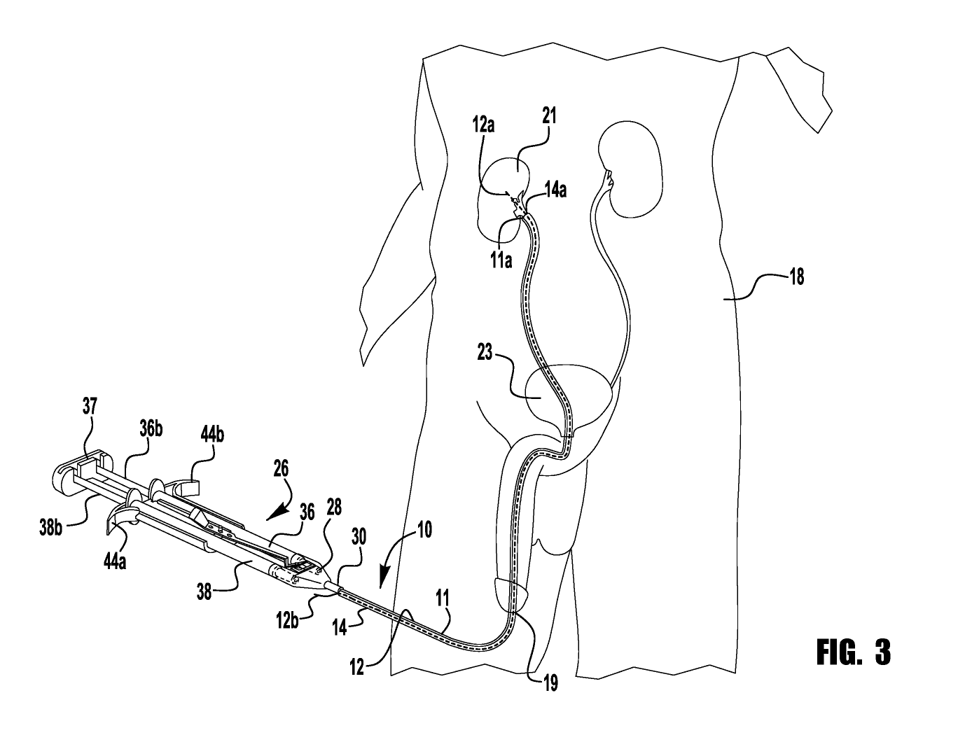

FIG. 1 illustrates a three-dimensional view of the catheter guide tube device 10, with a surgical wire 12 inserted therethrough. In general terms, the catheter guide tube device 10 includes a guide tube 14 is disposed adjacent to and adjoining a catheter 11, wherein the guide tube extends the length of the catheter. The catheter 11 is regulated and controlled with a catheter injection system 26. The first end 12a of the surgical wire 12 is designed to extend outward from a person's body, typically but not limited to a urethra, from the location where the catheter 11 is to be inserted into the body and ultimately located so that the end 11a of the catheter is at a desired location within the patient 18, as seen in FIG. 3, and described herein below. In another example the guide wire can be inserted through the patient's skin directly into an organ. The second end 12b of the surgical wire 12 is disposed within the person's body at the desired location within the patient 18.

The first end 12a of the surgical wire 12 is threaded into the first distal open end 14a of the guide tube 14 and the guide tube and attached catheter 11 are pushed onto the length of the surgical wire towards the second end 12b of the surgical wire 12. The catheter 11, being attached to the guide tube 14 is moved so that the distal end 11a is moved to the desired location within the patient 18. Upon reaching the second end 12b of the surgical wire 12 at the site within the patient 18, the distal end 11a of the catheter 11 reaches the desired location within the patient 18, as seen in FIG. 3.

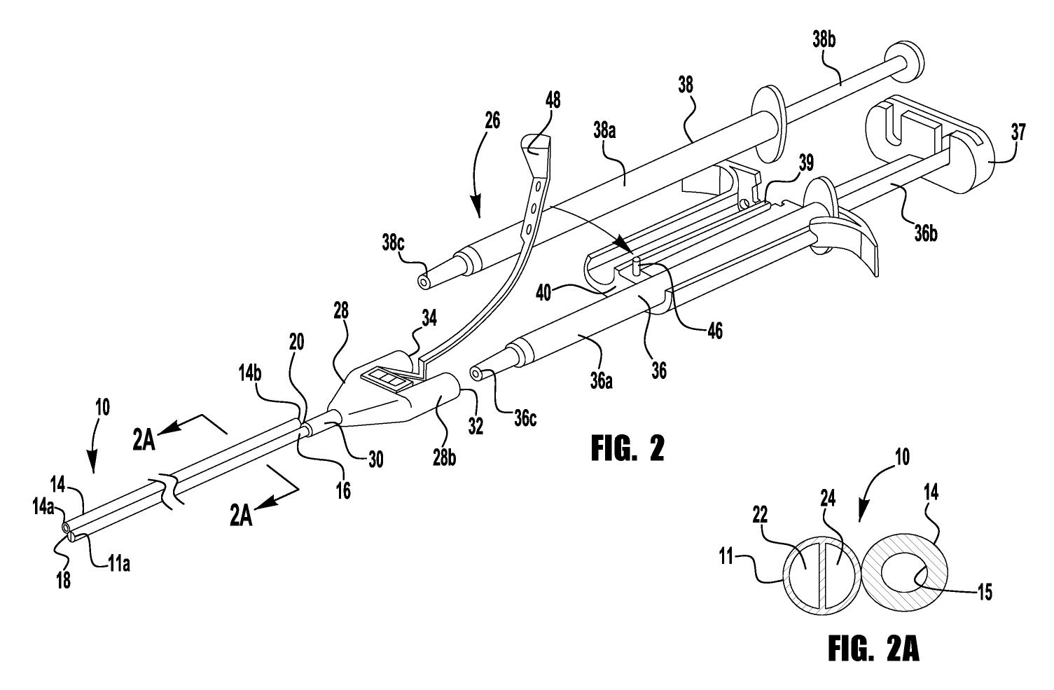

As seen in FIGS. 1 and 2, the improved catheter guide device 10, consisting of the guide tube 14 and the catheter 11, is typically made of a soft and biocompatible flexible material including distal end and proximal ends 18 and 20, such that it may stay inside body for a long time without causing any damages. The catheter guide device 10 may be of any desired appropriate length with a range in length between about 1 and 100 inches. Further, the catheter guide device 10 must have an inner diameter, with a range between about 0.025 centimeters (cm) to about 0.102 cm, such that it can be easily inserted into the patient 18 through urethra 19 without any risks of tearing the canals through which it is caused to pass or of causing traumas thereto.

As seen in FIG. 2, the guide tube 14 and the catheter 11 are fused together, in any desired fashion. This may include being molded from a single material, joined together with an adhesive, or any other desired and sterile method. The improved catheter guide device 10 is designed to be inserted into the body of the patient 17 to access the bladder, kidney, or any other appropriate location within the body.

The catheter 11 is typically composed of an elongated, flexible, cylindrical member which is of substantially uniform outside diameter throughout the entire length thereof. As illustrated in FIG. 2A, the catheter 11 may be divided into one or more segment portions 22 and 24. Traditionally, the proximal end 11b of the elongated, flexible, cylindrical member 11 is set in the form of a curl and is of denser material than the remainder of the elongated, flexible, cylindrical member in order to keep the curl shape. The catheter 11 is adapted to be shortened by removing a portion of the distal end 11a to tailor the overall length of catheter 11 to the needs of any specific patient 18.

The catheter 11 is regulated with catheter injection system 26. The catheter 11 connects at its proximal end 11b to a first end 28a of a coupler 28. Proximal end 11b of the catheter 11 is received in a rigid cuff 30 of the coupler 28, which bridges the catheter and the catheter injection system 26. There are two openings 32 and 34 at a second end 28b of the coupler 28, which receive one or more syringes 36 of injection system 26.

As illustrated, there are two syringes 36 and 38. Each syringe 36 and 38 has a hollow barrel 36a and 38a, respectively, fitted with a respective plunger 36b and 38b, respectively, and a narrow opening end 36c and 38c, respectively, which fit into the two openings 32 and 34 at a second end 28b of the coupler 28. In use, the plungers 36b and 38b dispel the liquid from the hollow barrels 36a and 38a, respectively, through the narrow openings 36c and 38c. The syringes 36 and 38 are shown as having respective hollow barrels 36a and 38a fitted with their respective plungers 36b and 38b. In the current embodiment, the plungers 36b and 38b are placed within a plunger mounting 37, allowing the plungers to be depressed simultaneously if desired. Alternatively, the plungers from each barrel may be joined together, so that they may be depressed simultaneously.

The syringes 36 and 38 may be molded as a single unit or may be separate syringes held together by clamp 39. As illustrated, the clamp 39 includes a main body 40 with two identical housing portions 42a and 42b. Each of the housing portions 42a and 42b are designed to temporarily secure the one or more syringes 36 and 38 while in use. The housing portions 42a and 42b are semi-circular and house the hollow barrels 36a and 38a of the syringes 36 and 38.

The clamp 39 includes a pair of handles 44a and 44b, disposed on either side of the clamp 39. Typically, a user may place a thumb on the plunger mounting 37, and a finger on the handles 44a and 44b to depress the plungers 36b and 38b of the syringes 36 and 38 to dispel the contents of the hollow barrels 36a and 38a through the narrow openings 36c and 38c, respectively.

As illustrated in FIGS. 1 and 2, there is a peg 46 that extends outward from the main body 40 of the clamp 39. Peg 46 is utilized to further secure the clamp 39 to the coupler 28 when the catheter injection system 26 is in use. A strap 48 with one or more openings 50 there through is attached to the coupler 28, whereby the peg 46 is designed to securely fit into one of the openings 50, and secure the coupler to the catheter injection system 26 when in use.

The guide tube 14 is fused together with the catheter 11, in any desired fashion. This may include being molded from a single material, joined together with an adhesive, or any other desired and sterile method. The guide tube 14 is typically composed of an elongated, flexible, hollow cylindrical member that is typically of substantially uniform outer and inner diameter throughout length of the entire hollow interior portion 15. Guide tube 14 is a cylindrical member having an internal diameter with a range between about between about 0.025 centimeters (cm) to about 0.102 cm. The guide tube 14 is typically of the same length as the catheter 11. The second proximal end 14b of guide tube 14 can extend to the proximal end 11b of the catheter 11, and may also be received in the rigid cuff 30 of the coupler 28. Alternatively, the second proximal end 14b may simply attach to the catheter 11 outside of the rigid cuff 30 of the coupler 28. The guide tube 14 is designed to assist a medical professional in guiding the catheter 11 to the correct site on the patient 18.

FIG. 3 illustrates a three-dimensional view of the improved catheter guide device 10 in use with the genital urethary system of a patient 18. A wire 12 is seen exiting the urethra 19 of a patient 18. As illustrated, a urological procedure is depicted, although the catheter guide tube 10 may be utilized in any appropriate medical operation. The wire 12 acts as a guide to properly dispose the distal end 1 la of the catheter 11 at the desired location within the patient 18. The wire 12 is inserted into the kidney 21 of the patient at the location adjacent to where the distal end 11a of the catheter 11 is required.

In operation, the surgical wire 12 is first placed through the genital urethary system such as through the urethra 19 of the patient 18 and into the kidney 21. While the description relates to directing a surgical wire to a kidney, it is equally applicable for use when the surgical wire is placed in some other organ or location in the patient's body where the catheter is required. The first end 12a of the surgical wire 12 is placed adjacently to the location on the kidney 21 where the catheter 11 is to be placed for the medical procedure. The distal end 18 of the improved catheter guide device 10 is inserted by a cystoscopic procedure through the urethra 19, and bladder 23 until reaching the kidney 21. It should be noted that the improved catheter guide 10 has a very reduced diameter, so that it can be easily introduced into urethra without tearing the same or causing traumas thereto.

To accomplish this, the second end 12a of the surgical wire 12 is inserted into the first distal open end 14a of the guide tube 14. The guide tube 14 s fed onto the surgical wire 12 through the urethra 19, and bladder 23. Then, the first distal open end 14a of the guide tube 14 reaches the kidney 21, whereby the distal end 11a of the catheter 11 simultaneously reaches the kidney as it is attached to the guide tube.

Once the catheter 11 is in place on the kidney 21, the wire guide 12 can be removed. The medical professional may then may place a thumb on the plunger mounting 37, and a finger on each of the handles 44a and 44b to depress the plungers 36b and 38b of the syringes 36 and 38 to dispel the contents of the hollow barrels 36a and 36b through the narrow openings 36c and 38c. The contents then flow through the catheter 11 to the desired location in the patient 18, in this case a location on the kidney 21. Then, the catheter guide device 10 may simply be removed from the urethra 19 of the patient.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A catheter guide tube device to assist in the insertion of a catheter through a genital urethary system to a desired location, characterized by;
a catheter;
a catheter guide tube being attached to the catheter; and
a catheter injection system to regulate and control a flow of liquid through the catheter.

2. The catheter guide tube device of claim 1 further characterized by a coupler having a first end connected to a distal end of the catheter a second end being connected to the catheter injection system.

3. The catheter guide tube device of claim 1 further characterized by the catheter guide tube being disposed adjacent to and adjoining the catheter.

4. The catheter guide tube device of claim 3 further characterized by the catheter guide tube being fused together with the catheter.

5. The catheter guide tube device of claim 4 further characterized by the catheter guide tube and the catheter being molded from a single material.

6. The catheter guide tube device of claim 4 further characterized by the catheter guide tube and the catheter being joined together with an adhesive.

7. The catheter guide tube device of claim 1 characterized by the catheter guide tube and the catheter having the same length.

8. The catheter guide tube device of claim 1 further characterized by the catheter guide tube being composed of an elongated, flexible, hollow cylindrical member.

9. The catheter guide tube device of claim 1 further characterized by the catheter guide tube having substantially uniform outer and inner diameter throughout the entire length thereof.

10. The catheter guide tube device of claim 1 further characterized by a surgical wire, adapted to he disposed within a patient's body from a location where the catheter is to be inserted into the patient's body and extending outwardly from the patient's body, whereby the surgical wire can be threaded through the catheter guide tube so that the catheter guide tube and attached catheter are pushed along the surgical wire towards a second end of the surgical wire so that the catheter being attached to the guide tube is moved so that the distal end of the catheter is moved to the desired location within the patient's body.

11. The catheter guide tube device of claim 2 further characterized by catheter being divided into two segment portions.

12. The catheter guide tube device of claim 11 further characterized by the catheter injection system having two syringes received within two openings at a second end of the coupler, wherein the flow of the liquid controlled and regulated by the catheter injection system is directed through the two segment portions of the catheter.

13. A method of inserting a catheter through a genital urethary system to a desired location in a patient's body, comprising the steps of;
inserting a surgical wire through the genital urethary system of the patient's body whereby a first end of the surgical wire is placed adjacently to a desired location within the patient's body adjacent to location the catheter is to he placed and a second end of the surgical wire remains external to the patient's body;
inserting the second end of the surgical wire into a first distal open end of a catheter guide tube attached to a catheter; and
feeding the catheter guide tube onto the surgical wire, wherein the first distal open end of the guide tube and a distal end of the catheter reaches the desired location within the patient's body.

14. The method of claim 13 further including removing the surgical wire after the first distal open end of the guide tube and the distal end of the catheter reaches the desired location within the patient's body.

15. The method of claim 14 further including directing the flow of liquid through the catheter to the reaches the desired location within the patient's body.

16. The method of claim 15 further including connecting a catheter injection system to a proximal end of the catheter to direct the flow of liquid through the catheter.

17. The method of claim 16 further including dividing the catheter into two segment portions.

18. The method of claim 17 further including providing the catheter injection system with two syringes containing the liquid and a plunger on each of the syringes for dispelling the liquid within the syringes.

19. The method of claim 18 further including depressing the plungers on the syringes to dispel the liquid within the syringes into the two segment portions of the catheter to the desired location in the patient's body.

20. The method of claim 19 further including removing the guide tube and the catheter 18, from the desired location in the patient's body.
